(19) United States
(12) Patent Application Publication
(10) Pub. No.: US 2015/0282789 A1
(43) Pub. Date: Oct. 8, 2015

(54) Closure Device

(71) Applicant: ENDOHEART AG, Winterthur (CH)

(72) Inventor: Christoph Hans Huber, Bern (CH)

(73) Assignee: ENDOHEART AG, Winterthur (CH)

(21) Appl. No.: 14/439,399

(22) PCT Filed: Oct. 29, 2013
(86) PCT No.: PCT/CH2013/000189

§ 371 Date: Apr. 29, 2015

(30) Foreign Application Priority Data
Oct. 29, 2012 (CH) 2168/12

(51) Int. Cl. A61B 17/00; A61B 19/00; A61N 1/05

(52) U.S. Cl. CPC A61B 17/0057; A61N 1/0587; A61B 19/54; A61B 2017/00592; A61B 2017/00619; A61B 2017/00672; A61B 2017/00889; A61B 2017/00893; A61B 2017/00898; A61B 2019/5466; A61B 2019/5425

(57) ABSTRACT

Apparatus and methods for closing an access hole in a heart. The apparatus may include a heart plug that may include an absorbent mass that applies pressure to myocardial tissue in the access hole by absorption of fluid. The mass may have a length selected to match a length of the hole or to exceed the length of the hole by a predetermined amount. The mass may have an unexpanded state. The mass may have an expanded state. The expanded state may be a state in which the plug is partially saturated or saturated with a fluid. The mass may have an unexpanded state diameter that is selected for both rapid occlusion of the hole and sliding clearance within a delivery catheter having an outside diameter sized for traversal of the hole. The mass may include a matrix. The matrix may define interstitial space for absorption of the fluid.

### CROSS-REFERENCE TO OTHER APPLICATIONS

This application claims priority under Title 35, U.S.C. Sec. 119, from Swiss Patent Application No. 02168/12, which was filed on Oct. 29, 2012, and is hereby incorporated by reference herein in its entirety.

### BACKGROUND

A closure device is typically used to occlude an access hole that is surgically opened to facilitate a therapeutic cardiac or vascular procedure. The closure device is used to seal off the access hole in order to re-establish the integrity of an accessed organ wall or vessel. A closure device can also be used to close anatomical defects such as a septal defect in an atrial septum. Typical closure of an access hole involves the use of sutures or large closure devices. Use of sutures or large closure devices requires disturbance of skin and other tissue along the access path to the access hole. The disturbance may be greater than the disturbance required to perform the therapeutic procedure.

It would be desirable, therefore, to provide apparatus and methods for closing an access hole that do not require disturbance greater than that required to perform the therapeutic procedure.

### BRIEF DESCRIPTION OF THE DRAWINGS

The objects and advantages of the invention will be apparent upon consideration of the following detailed description, taken in conjunction with the accompanying drawings, in which like reference characters refer to like parts throughout, and in which:

FIG. 1 is an illustrative cross-sectional view of apparatus in accordance with the principles of the invention along with illustrative anatomy in connection with which the invention may be practiced;

FIG. 2 is an illustrative cross-sectional view of the apparatus shown in FIG. 1 along with the anatomy shown in FIG. 1 when the apparatus is in a different state from that shown in FIG. 1;

FIG. 3 is an illustrative cross-sectional view of the apparatus shown in FIG. 2 along with the anatomy shown in FIG. 2 when the apparatus is in a different state from that shown in FIG. 2;

FIG. 4 is an illustrative cross-sectional view of the apparatus shown in FIG. 3 along with the anatomy shown in FIG. 3 when the apparatus is in a different state from that shown in FIG. 3;

FIG. 5 is an illustrative cross-sectional view of the apparatus shown in FIG. 4 along with the anatomy shown in FIG. 4 when the apparatus is in a different state from that shown in FIG. 4;

FIG. 6 is an illustrative cross-sectional view of the apparatus shown in FIG. 5 along with the anatomy shown in FIG. 5 when the apparatus is in a different state from that shown in FIG. 5;

FIG. 7 is an illustrative cross-sectional view of the apparatus shown in FIG. 6 along with the anatomy shown in FIG. 6 when the apparatus is in a different state from that shown in FIG. 6;

FIG. 8 is an illustrative perspective view of other apparatus in accordance with the principles of the invention;

FIG. 9 is an illustrative perspective view of parts of yet other apparatus in accordance with the principles of the invention along with other anatomy, in partial cross section, in connection with which the invention may be practiced;

FIG. 10 is an illustrative partial cross-sectional view of still other apparatus, in accordance with the principles of the invention, along with the anatomy shown in FIG. 5, taken from a direction like that shown by lines 10-10 (shown in FIG. 5) of the apparatus shown in FIG. 5;

FIG. 11 is an illustrative cross-sectional view of still other apparatus in accordance with the principles of the invention;

FIG. 12 is an illustrative perspective view of still other apparatus in accordance with the principles of the invention;

FIG. 13 is an illustrative perspective view of still other apparatus in accordance with the principles of the invention;

FIG. 14 is an illustrative perspective view of still other apparatus in accordance with the principles of the invention;

FIG. 15 is an illustrative partial cross-sectional, partial perspective view of still other apparatus in accordance with the principles of the invention along with the anatomy shown in FIG. 1; and

FIG. 16 is an illustrative perspective view of still other apparatus in accordance with the principles of the invention.

### DETAILED DESCRIPTION

Apparatus and methods for closing an access hole in a heart, blood vessel or other anatomy are provided. The apparatus may include a heart plug. The heart plug may include an absorbent mass that is configured to apply pressure to myocardial tissue in the access hole by absorbing a fluid. The pressure may create friction-based anchoring of the plug in the access hole.

The mass may have a longitudinal axis. The mass may have a central axis. The longitudinal axis may coincide with the central axis. The mass may have a radial direction. The radial direction may be perpendicular or substantially perpendicular to the axis.

Wetting of the plug by the fluid may cause the plug to expand. The plug may expand radially. The plug may expand longitudinally. Liquid-swollen plug material may expand until the expansion force of the plug equals the resistance of the surrounding tissue. When the expansion force equals the resistance of the surrounding, a friction-based anchoring may be created.

Stored elastic energy in material in the plug may contribute to expansion when the plug interacts with fluid. Material in the plug may absorb the fluid by wicking. The wicking may contribute to the expansion. The expansion may continue until the plug is constrained by surrounding anatomy. The expansion may continue until expansion forces generated by the wicking are balanced by external forces from the anatomy on the plug. The plug may be a plug that does not include a non-expandable cover.

If the plug is compressed, for example by heart contraction, when the fluid is present in the plug, the plug may resist the compression. Elastic properties of material in the plug may produce some or all of the resistance. Some or all of the resistance may be generated by wetting of the plug material by the fluid. Some or all of the resistance may be generated by viscous resistance to expulsion of a fraction of the fluid through the material. Some or all of the resistance may be generated by frictional forces acting between fibers or other elements inside the plug. Some or all of the resistance may be generated by contact forces acting between fibers or other elements inside the plug.

When the compression is reversed, for example by heart relaxation, the plug may rebound. The plug may rebound based on one or more of the aforementioned mechanisms that provide expansion, resistance to compression or by any other suitable mechanism. For example, wicking may contribute to the rebound. Restoration of equilibrium between surface forces at the contact between the fluid and the plug material may contribute to the rebound. A pressure drop in the plug as the plug is expanded from reversal of the compression may contribute to the rebound.

When saturated or partially saturated with the fluid, the plug may be resistant to compression and retain its seal against the heart wall. When saturated or partially saturated with the fluid, the plug may be compliant and avoid causing stress concentrations that may injure the heart wall. When saturated or partially saturated with the fluid, the plug may be both resistant to compression and compliant.

The plug may efficiently close the access hole. The plug may provide a rapid seal. The plug may provide a permanent seal. The plug may provide an atraumatic seal. The plug may restore anatomical integrity to the organ in which the access hole is disposed. The plug may restore functional integrity to the organ in which the access hole is disposed.

The plug may be self-sealing. The plug may be self-anchoring. The plug may be self-centering in a plane transverse or perpendicular to the access hole.

When the hole is in the heart, the hole may be at an apex of the heart. For example, the hole may be at a ventricular apex of the heart.

The plug may include any suitable biocompatible material. The biocompatible material may be self-expanding. The biocompatible material may be self-anchoring to heart tissue.

The plug may include any suitable bioabsorbable material. The bioabsorbable material may be self-expanding. The bioabsorbable material may be self-anchoring to heart tissue.

The plug may include any suitable biodegradable material. The biodegradable material may be self-expanding. The biodegradable material may be self-anchoring to heart tissue.

The mass may have an unexpanded state. The mass may have an expanded state. The expanded state may be a state in which the plug is partially saturated with a fluid. The expanded state may be a state in which the plug is saturated with the fluid. The fluid may be blood, water, saline solution, plasma or any other suitable fluid.

The mass may have an unexpanded state diameter that is selected for rapid occlusion of the hole. The mass may have an unexpanded state diameter that is selected for sliding clearance within a delivery catheter having an outside diameter sized for traversal of the hole. The mass may have an unexpanded state diameter that is selected for both rapid occlusion of the hole and sliding clearance within a delivery catheter having an outside diameter sized for traversal of the hole.

The mass may have a length that is selected to match the length of the hole. The selected length may be a length of the mass when the mass is unexpanded. The selected length may be an expected length of the mass when the mass is subsequently expanded. The selected length may be selected to match a length that is less than the length of the hole. The mass may have a length selected to match a length that is greater than the length of the hole. The mass may be positioned so that the mass extends out of a distal end of the hole. The mass may be positioned so that the mass extends out of a proximal end of the hole. When the mass is positioned to extend out of the distal end of the hole, the mass may extend radially outward to a radius greater than the radius of the hole. The mass may form a shape like that of a champagne bottle cork. The greater radius may provide anchoring by interference with an annular region of heart tissue adjacent a distal end of the hole.

The mass may include a growth factor treatment. The treatment may be applied to a surface of the mass.

The mass may include any suitable biocompatible material. The mass may include any suitable bioabsorbable material. The mass may include any suitable biodegradable material.

The mass may include a matrix. The matrix may define interstitial space for absorption of the fluid.

The plug may include an antibiotic pharmacological agent. The antibiotic agent may be supported by the matrix. The heart plug may include a polymerizing agent. The polymerizing agent may be configured to polymerize a blood constituent. The polymerizing agent may be supported by the matrix. The polymerizing agent may be configured to provide a polymerized blood constituent in the interstitial space to give the plug elastic properties.

The heart plug may include a photoactivated compound. The compound may include powder. The compound may be provided in a coating on the mass. The compound may be activated by applying light through a catheter. When activated in the presence of the fluid, the compound may provide the plug with elastic properties.

The matrix may include nonwoven material. The matrix may include fibrous matter. The fibrous matter may include cellulose. The fibrous matter may include cotton. The fibrous matter may include rayon. The fibrous matter may include polyester. The fibrous matter may include polyethlyne. The fibrous matter may include any other suitable material.

The fibrous matter may be prepared in a manner that provides for expansion shortly after initial contact with the fluid. Apparatus and methods for such preparation are set forth, for example, in U.S. Pat. Nos. 6,310,269 and 6,748,634, which are hereby incorporated herein in their entireties.

The matrix may include a gelatinous material.

The matrix may define a porous polymer network.

The heart plug may include a cannula. The cannula may be a permanent part of the heart plug. The permanent cannula may be non-removable in the sense that it is allowed to remain in the plug when the plug is permanently deployed in the heart. The cannula may be removable from the heart plug. The cannula may extend through the mass. The cannula may extend along the entire longitudinal axis of the mass, thereby passing through the mass. The cannula may extend partially through the mass. The cannula may be configured to receive a guide wire. The cannula may extend partially through the mass.

The heart plug may include a carriage loop. The loop may extend away from an outer surface of the mass. The loop may be configured to receive a guide wire. The guide wire may be used as a “tandem wire,” along which the plug may slide, with the tandem wire offset from the central axis of the plug. The tandem wire may be at a greater radial distance from the central axis than is the outer surface of the plug.

The heart plug may include a porous veiling about the mass. The mass may be configured to press the veiling against the myocardial tissue when the mass expands. The mass may be configured to press the veiling against the myocardial tissue in response to absorption of the fluid by the mass.

The veiling may include any suitable biocompatible material. The veiling may include any suitable bioabsorbable material. The veiling may include any suitable biodegradable material. The veiling may include nonwoven material. The veiling may include cellulose. The veiling may include polyester. The veiling may include polyethylene.

The mass may have a dry diameter. The mass may have a wet diameter. The wet diameter may be a saturated diameter. The wet diameter may be a partially saturated diameter. The wet diameter may be greater than the dry diameter. The veiling may be nonexpendable relative to the mass. The veiling may be expandable. When the veiling is nonexpandable relative to the mass, the veiling may have a maximum diameter.

The access hole may have a diastolic diameter when the heart is relaxed. The saturated diameter of the mass may be greater than the diastolic diameter of the hole. The maximum diameter of the veiling may be greater than the wet diameter of the mass. The maximum diameter of the veiling may be greater than the wet diameter of the mass when the mass is fully expanded by absorption.

The plug may be provided in different compressed diameters corresponding to different access hole diameters. The plug may be provided in different compressed lengths corresponding to different access hole lengths. One or more plugs of different diameters or lengths may be provided in a kit.

The plug may include an anchor. The anchor may be one of a plurality of anchors. Each of the plurality of anchors may have one or more features in common with the other anchors. The anchor may have a base. The base may be affixed to the mass. The anchor may be affixed to an outer radial surface of the mass. The anchor may be affixed to the distal end of the mass. The anchor may be affixed to the proximal end of the mass. The anchor may be affixed to the veiling.

The anchor may have an engagement end. The engagement end may be configured to engage the heart. The engagement end may be configured to atraumatically engage heart muscle. The anchor may be any suitable anchor. The engagement end may include a piercing tip that is supported by a stem that points radially outward and proximal from the base. The engagement end may extend, for example, up to about 0.5 mm, along the stem, from the base. The engagement end may extend, for example, up to about 1 mm, along the stem, from the base. The engagement end may extend, for example, up to about 2 mm, along the stem, from the base. The piercing tip may thus engage the myocardium upon deployment of the plug. The piercing tip may be pin-like. The piercing tip may thus pierce the myocardium by shifting the plug in the proximal direction.

The anchor may have the form of a link of a chain. Alternating interlinked links may present a radially outward protrusion to the myocardium. The links may provide traction on the myocardium in a manner similar to the way traction chains for vehicle tires provide traction on travel surfaces.

The anchor may be one of a plurality of anchors. The plurality of anchors may be arranged about the surface of the plug to engage the myocardial tissue when the plug is deployed in the access hole. The anchors may be linked to each other by a girdle that encircles or partially encircles the plug. The plug may include one or more girdles of anchors.

The plug may include an imaging marker. The marker may extend from a distal portion of the mass. The marker may be configured to signal registration of the distal portion with an orifice at a distal end of the hole. The marker may be radiopaque. The marker may be selected for acoustic reflection contrast. The marker may be selected for magnetic resonance imaging contrast. The marker may provide a visual aid for positioning the plug in the access hole. The marker may provide a visual aid for delivering the plug to the access hole. The marker may be distributed about the surface, or within the volume, of the plug in a patterned fashion such that an expanded portion of the plug may be visually distinguished from an unexpanded portion of the plug. For example, the marker may include a lobed thread that encircles or partially encircles the plug and expands with the plug. A marker thread may be formed from a girdle of anchors.

The heart plug may include a cap. The heart plug may include a non-thrombogenic cap. The cap may include an umbrella form. The cap may include a disc form. The cap may extend, at a distal end of the mass, radially away from a central axis of the mass. The cap may include an elastomeric material, an alloy such as that available from Nitinol Devices & Components, Inc., Fremont Calif., under the trade name “Nitinol,” a surgical steel, a surgical steel with non-thrombogenic coating, a polymeric material, a film, a membrane or any other suitable material. The cap may be attached to the distal end of the mass. The cap may be pinned to the distal end of the mass. The cap may be glued to the distal end of the mass. The cap may be sutured to the distal end of the mass. The cap may include biocompatible material. The cap may include bioabsorbable material. The cap may include biodegradable material. The cap may include material that is similar to or the same as material that is included in the veiling.

The mass may have a first diameter when the mass is in a compressed state. The mass may have a second diameter when the mass is in an expanded state. The cap may have a cap diameter that is greater than the first diameter. The cap may be directly affixed to a cannula. The cap may extend away from the cannula. The cannula may extend substantially along a central axis of the plug from a proximal end of the plug to a distal end of the plug. The cannula may be configured to receive a guidewire.

The cap diameter may be about the same size as the second diameter.

The plug may include a proximal cap. The proximal cap may extend, at a proximal end of the mass, radially away from the central axis.

The proximal cap may be directly affixed to the cannula. The proximal cap may extend away from the cannula.

The distal cap may be used to anchor the plug against heart tissue near the distal end of the access hole. The proximal cap may be used to anchor the plug against heart tissue near the proximal end of the access hole.

The distal cap may prevent the fluid from contacting the mass. The distal cap may prevent the fluid from contacting the mass when the mass is in the delivery catheter. The distal cap may prevent the fluid from contacting the mass when the mass or a portion of the mass is positioned inside a chamber of the heart. The distal cap may prevent the fluid from contacting the mass until the mass is positioned in the access hole. The distal cap may thus prevent the mass from expanding before the mass is desirably positioned in the access hole.

The plug may include an electrically conductive member. The plug may include an electrode. The plug may include a conductive lead. The veiling may support the electrode. The electrode may be configured to electrically engage the tissue. The lead may have a first terminal. The first terminal may be connected to the electrode. The first terminal may be connected to a second terminal. The second terminal may provide communication to a device external to the heart.

The electrically conductive member may be supported by the elongated member. The electrically conductive member may be configured to deliver to the heart wall a current that modifies a contraction frequency of the heart. The apparatus may include one, two, three, four, 10 or more, or any suitable number of electrically conductive members. The electrically conductive member may be an electrode.

The electrically conductive member may be used to provide current to the heart in conjunction with another electrically conductive member that is placed elsewhere in the heart, on the heart, or on the patient's skin and also provides pacing current to the patient's tissue.

The elongated member may include any suitable biocompatible material such as polymer, stainless steel, nickel titanium alloy or any other suitable material.

The apparatus may include, for each electrically conductive member, a current supply lead. The current supply lead may receive one or more cardiac pacing signals from a cardiac pacing signal generator. A connector may be provided for placing the current supply lead in electrical communication with the cardiac pacing signal generator. The cardiac pacing signal generator may include any suitable pacing device.

In some procedures, more than one of the apparatus may be used together. For example, a first instrument having electrically conductive members for transferring pulses to the heart and a second instrument having electrically conductive members for transferring pulses to the heart may be coaxially arranged, the first inside the second. The first instrument may be extend from the distal end of the second instrument and be advanced into the myocardium to perform a first procedure. During the first procedure, pulses may be transferred to the heart from the first instrument.

After the first procedure, the second instrument may be advanced along the first instrument into the myocardium. When the second instrument advances into the myocardium, pulses may be transferred to the heart from the second instrument. A current switch may be provided to transfer electrical energy from the first instrument to the second instrument. The current switch may analyze an electrical characteristic of one or both of the first and second instruments to detect the succession of the second instrument in the access opening. The current switch may deactivate the first instrument and activate the second instrument upon or about the time of the succession. The electrical characteristic may include a continuity. The electrical characteristic may include an impedance.

The electrically conductive member may be configured to provide to the heart wall a series of pulses. The pulses may be quantified by pacing parameters. The pacing parameters may include voltage, current, energy, duration, pulse frequency, maxima and minima thereof, and any other suitable pacing parameters.

Table 1 shows illustrative ranges of some pacing parameters.

TABLE 1
Illustrative ranges of pulse current.

| Current | | Pulse | | Pulse | |
|---|---|---|---|---|---|
| From | To | duration | | frequency | |
| about | about | From about | To about | From about | To about |
| (mA) | (mA) | (ms) | (ms) | (Hz) | (Hz) |
| 0.01 | 0.05 | 0.01 | 0.05 | 1 | 3 |
| 0.05 | 0.1 | 0.05 | 0.1 | 3 | 10 |
| 0.1 | 1 | 0.1 | 1 | 10 | 30 |
| 1 | 3 | 1 | 3 | 30 | 60 |
| 3 | 10 | 3 | 10 | 60 | 100 |
| 10 | 20 | 10 | 20 | 100 | 130 |
| 20 | 30 | 20 | 40 | 130 | 160 |
| 30 | 40 | 40 | 60 | 160 | 200 |
| 40 | 50 | 60 | 80 | 200 | 230 |
| 50 | 60 | 80 | 100 | 230 | 260 |
| 60 | 100 | 100 | 200 | 260 | 300 |

Each pulse may carry from about 0.1 to about 40 milliamp (“mA”). Each pulse may have a duration that is in the range of about 0.1 to about 100 millisecond (“ms”). The pulses may be delivered with a frequency of about 10 to about 300 pulses per second.

The electrically conductive member may include copper, silver, gold, platinum, polymer or any other suitable conductive material. The electrically conductive member may include conductive wire, tape, foil, sheet, rod, bar, tube, shot or any other suitable form.

The electrically conductive member may be configured to be in indirect contact with the heart wall.

The electrically conductive member may be configured as an antenna. The antenna may sense a native cardiac electric field in a chamber on the interior side of the heart wall. The antenna may communicate a signal that corresponds to the field to a receiver exterior the heart wall. The receiver may be part of an electrocardiograph device. The antenna may communicate the cardiac signal via a transmission line. The antenna may communicate the cardiac signal wirelessly.

The apparatus may include a pressure sensor. The pressure sensor may be supported by the mass. The pressure sensor may be supported by the electrically conductive member. The pressure sensor may be configured to sense a pressure in the chamber. The pressure sensor may be configured to sense a pressure in the heart wall. The pressure sensor may be configured to sense a pressure in the access hole. The pressure sensor may be configured to transmit a corresponding pressure signal to a receiver exterior the heart wall. The antenna may communicate the pressure signal via a transmission line. The antenna may communicate the pressure signal wirelessly.

The apparatus may include a chemical sensor. The chemical sensor may be supported by the mass. The chemical sensor may be supported by the electrically conductive member. The chemical sensor may be configured to measure chemical values such as, for example, pH, lactate, cardiac enzymes, electrolytes. The chemical sensor may be configured to transmit a corresponding signal to a receiver exterior the heart wall. The chemical sensor may transmit the chemical signal via a transmission line. The chemical sensor may transmit the chemical signal wirelessly.

The chemical sensor may be calibrated to sense a chemical species. The species may be present in a chamber interior the heart wall. The species may be present at a myocardial tissue surface that is exposed in a heart wall access opening and transmit a corresponding chemical signal to a receiver exterior the heart wall.

The chemical sensor may detect the chemical value based on conductivity of the heart wall. The chemical sensor may detect the chemical value based on capacitance of the heart wall. The chemical sensor may detect the chemical value based on an electrical potential of the heart wall. The chemical sensor may include a porous layer. The chemical sensor may detect the chemical value based on conductivity of the porous layer. The chemical sensor may detect the chemical value based on capacitance of the porous layer. The chemical sensor may detect the chemical value based on an electrical potential of the porous layer.

The apparatus may include a processor that is configured to change a pacing parameter, for example, a frequency of the current, based on the native cardiac signal. The processor may be configured to change the pacing parameter based on one or more pressure signals. The processor may be configured to change the pacing parameter based on one or more chemical signals.

The electrically conductive member may be configured to be released from the mass and inserted in the heart wall. The electrically conductive member may be inserted into the endocardium. The electrically conductive member may be inserted into the myocardium. The electrically conductive member may be inserted into the pericardium. The electrically conductive member may be fixed onto the endocardium. The electrically conductive member may be fixed onto the myocardium. The electrically conductive member may be fixed onto the pericardium.

The electrically conductive member may be anchored in the heart wall. The electrically conductive member may be anchored by a barb, a coil or any other suitable anchor. The electrically conductive member may be a wire. The wire may have a distal end that is driven into the heart wall. The electrically conductive member may be configured to be released from the elongated member and placed on the heart wall. The electrically conductive member may be left in place in the heart wall after removal of the elongated member from the access opening. The electrically conductive member may later be removed from the heart.

The apparatus may include a conductor that is attached to the electrically conductive member and runs proximally from the electrically conductive member through a lumen of a delivery device.

The plug may include, for each electrode, a current supply lead. The current supply lead may receive one or more cardiac pacing signals from a cardiac pacing signal generator. A connector may be provided for placing the current supply lead in electrical communication with the cardiac pacing signal generator. The cardiac pacing signal generator may include any suitable pacing device.

The mass may include a stem that extends between the distal end and the proximal end. The mass may include a shaft that extends between the distal end and the proximal end. The shaft may have one or more features in common with the stem. The stem may have a first diameter. The distal end may have a second diameter. The second diameter may be greater than the first diameter. The proximal end may have a third diameter. The third diameter may be greater than the first diameter.

The electrode may discharge from the distal end. The electrode may discharge from the proximal end. The electrode may discharge from the stem. The electrode may discharge from the shaft.

The mass may include an electrical energy storage source such as a battery. The mass may include a pacing signal generator. The battery may supply electrical current to the electrodes. The signal generator may control the current so that the current is provided in a therapeutic form.

The battery may be separate from the mass. The battery may be separately implantable in the patient. When the battery is implanted separately from the mass, the battery may be in wired electrical communication with the mass.

The battery may be inductively recharged from a source exterior the patient.

The apparatus may include a processor that is configured to change a pacing parameter, for example, a frequency of the current based on the native cardiac signal. The processor may be configured to change the pacing parameter based on one or more pressure signals. The processor may be configured to change the pacing parameter based on one or more chemical signals.

The plug may include a closure device for percutaneous insertion. The plug may include a closure device for surgical implantation. The closure device may expand from a smaller diameter to a larger diameter. The closure device may be embodied as a shaft that expands from a smaller diameter to a larger diameter. The shaft may include an inner lumen. The shaft may have a proximal end and a distal end. One or both of the ends may include an umbrella or anchor like structure. The shaft may include radiopaque markers. The radiopaque markers may be on the outer surface of the shaft. The radiopaque markers may be in the inside of the shaft. The radiopaque markers may be both on the outer surface of and inside the shaft. The radiopaque markers may be the umbrella or anchor like ends. The shaft expansion may initiate automatically by interaction with the fluid.

The shaft may be made from biocompatible cellulose or cellulose-like material having haemostatic properties. The shaft may be made from any biocompatible material able to be swollen when wetted and having hemostatic properties. The shaft may be self-anchoring to surrounding tissue.

The shaft may have an outer lining. The outer lining may improve anchoring. The outer lining may improve healing of the surrounding tissue. The outer lining may facilitate ingrowth of the surrounding tissue.

The shaft may work like a plug to immediately seal off the opening in the heart wall. The shaft may be bio-absorbable. The shaft may be biodegradable.

Wire-guided delivery of the closure device may be achieved percutaneously.

The closure device may include a pacing electrode. The pacing electrode may be temporary. The pacing electrode may be removed from the in-situ closure device.

Delivery of the closure device may be accomplished using an access device.

The methods may include a method for occluding the access hole. The methods may include introducing the plug into the access hole; positioning the plug adjacent myocardial tissue that is exposed in the hole; and releasing the plug so that the plug, by exerting a traction on the tissue, resists being dislodged from the hole by systolic blood pressure.

The positioning may include placing the plug in direct contact with the tissue such that the traction is transmitted through the contact and not through an anchor. The placing may include distributing contact between the plug and the tissue so that substantially all of the tissue is contacted by the plug. The placing may include distributing contact between the plug and the tissue so that substantially all of the tissue in a length of the hole is contacted by the plug.

The length may define a distal portion of the hole. The length may define a proximal portion of the hole.

The length may range from about 5% to about 10% of the length of the hole, from about 5% to about 10% of the length of the hole, from about 11% to about 20% of the length of the hole, from about 21% to about 30% of the length of the hole, from about 31% to about 40% of the length of the hole, from about 41% to about 50% of the length of the hole, from about 51% to about 60% of the length of the hole, from about 61% to about 70% of the length of the hole, from about 71% to about 80% of the length of the hole, from about 81% to about 90% of the length of the hole, from about 91% to about 100% of the length of the hole. The plug may have a length that is from about 100% to about 105% of the length of the hole. The plug may have a length that is from about 106% to about 110% of the length of the hole. The plug may have any other suitable length.

The introducing, the positioning and the releasing may be completed in a period that has a duration that is less than a duration of time required for a clotting cascade to cause clotting material to sufficiently engage the plug with the tissue to resist the systolic pressure.

The method may include expanding the plug by injecting the fluid into the plug.

The plug may be formed substantially in situ by injecting foam into a space defined by the distal cap, the proximal cap and the myocardial tissue.

The method may include injecting the polymerizing agent into the plug. The method may include injecting the photoactive compound into the plug.

The injecting may include transforming blood, absorbed in a matrix of the plug, into a solid.

The systolic pressure may be in the range from about 60 to about 80 mm Hg. The systolic pressure may be in the range from about 81 to about 100 mm Hg. The systolic pressure may be in the range from about 101 to about 120 mm Hg. The systolic pressure may be in the range from about 121 to about 140 mm Hg. The systolic pressure may be in the range from about 141 to about 160 mm Hg. The systolic pressure may be in the range from about 161 to about 180 mm Hg. The systolic pressure may be in the range from about 181 to about 200 mm Hg. The systolic pressure may be in the range from about 201 to about 225 mm Hg. The systolic pressure may be in the range from about 226 to about 250 mm Hg.

The systolic pressure may be greater than 100 mm Hg. The systolic pressure may be greater than 120 mm Hg. The systolic pressure may be greater than 160 mm Hg. The systolic pressure may be greater than 180 mm Hg. The systolic pressure may be greater than 250 mm Hg.

The introducing may include delivering the plug percutaneously. The delivering may include passing the plug through an epidermal incision having a length no greater than about one centimeter.

The method may include, after the releasing, permanently closing the epidermal incision.

The absorbent mass may be an absorbent core. The releasing may include deploying the absorbent core and the porous veiling, disposed about the core and configured to be displaced against the myocardial tissue by expansion of the core.

The introducing may include providing at a distal end of the plug a cap. The cap may extend radially away from a cylindrical axis of the plug. The cylindrical axis may be the central axis. The cap may be configured to retain fibers of the plug when the plug is in contact with the fluid. The cap may thus reduce the likelihood of the fluid entraining fibers as the fluid flows near the cap.

The introducing may include advancing the plug along a guidewire that passes through the access hole. The advancing may involve an over-the-wire arrangement. The advancing may involve a tandem wire arrangement.

The plug may be configured to seal, by absorption of the fluid, a lumen that is configured to translate along the guide wire.

The releasing may include disengaging a coupling between a proximal end of the plug and a distal end of a delivery wire. The delivery wire may be a wire that is not a guide wire.

The positioning may include advancing the plug distally in the hole, along a catheter lumen, using a pusher.

The pusher may be a pusher that is not coupled to the plug. For example, the pusher may be a pusher that is not configured to pull the plug proximally.

The positioning may include moving the plug proximally in the hole by moving a delivery catheter proximally in the hole.

The introducing may include providing on a distal end of the plug an articulating radiopaque marker that mechanically signals detection of an orifice of the hole at the distal end of the hole, interior to the heart. For example, the marker may signal detection of the orifice by deforming, being displaced or being rotated by an edge of the orifice as the plug is drawn proximally through the hole.

The releasing may include aligning a distal end of the plug with the internal orifice.

The positioning may include deploying a distal cap that extends, at a distal end of the plug, radially away from a central axis of the plug. The distal cap may include fluoroscopically or acoustically detectable material.

The deploying may include sliding the distal cap along a guide wire. The releasing may include deploying a proximal cap that extends, at a proximal end of the plug, radially away from a central axis of the plug. The proximal cap may include fluoroscopically or acoustically detectable material.

The deploying may include sliding the distal cap along the guide wire.

The method may include engaging a distal cap of the plug against endocardial tissue adjacent the hole interior the heart.

The method may include delivering electrical current to a conductor held against the tissue by expansion of the plug. The delivering may include controlling a heart rhythm. The method may include receiving from the conductor current corresponding to a heart rhythm. The method may include receiving from the plug an electrical signal indicative of a position of the plug in the hole. The signal may indicate the position based on a resistance measurement. The signal may indicate the position based on an impedance measurement. The signal may indicate the position based on a capacitance measurement. The method may include providing an electrical excitation to the plug before receiving the electrical signal. The signal may indicate the position based on time-domain reflection of an excitation signal.

The releasing may include deploying a distal end of the plug distal a distal orifice of the hole so that the distal end expands to a diameter greater than a diameter of the orifice.

Apparatus and methods in accordance with the invention will now be described in connection with the Figures. The features are illustrated in the context of selected embodiments. It will be understood that features shown in connection with one of the embodiments may be practiced in accordance with the principles of the invention along with features shown in connection with others of the embodiments.

Apparatus and methods described herein are illustrative. Apparatus and methods of the invention may involve some or all of the features of the illustrative apparatus and/or some or all of the steps of the illustrative methods. The steps of the methods may be performed in an order other than the order shown and described herein. Some embodiments may omit steps shown and described in connection with the illustrative methods. Some embodiments may include steps that are not shown and described in connection with the illustrative methods.

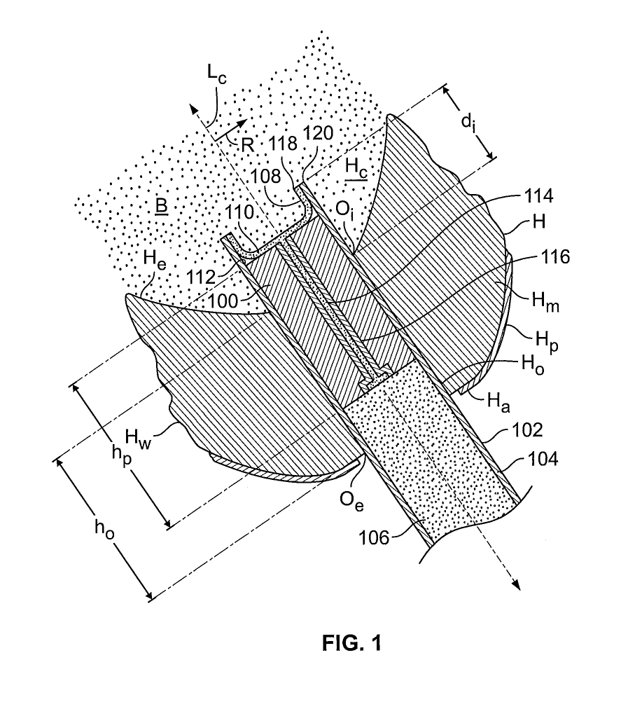

The apparatus and methods of the invention will be described in connection with embodiments and features of illustrative heart treatment devices and associated hardware and instrumentation. The device and associated hardware and instruments will be described now with reference to the FIG. 1t is to be understood that other embodiments may be utilized and structural, functional and procedural modifications may be made without departing from the scope and spirit of the present invention.

FIG. 1 schematically shows illustrative plug 100 for occlusion of an access hole such as Hₒ in a heart such as heart H. The occlusion may be performed on anatomy in or about heart H. The occlusion may be performed on anatomy in or about a chamber of heart H such as chamber H_{c}. Chamber H_{c} may be a left ventricle, a right ventricle, a left atrium or a right atrium. The occlusion may be performed on vasculature in or about heart H or on any other structure in or about heart H. Heart H may contract at a frequency.

Heart H may include pericardium Hₚ, myocardium Hₘ and endocardium Hₑ. Heart H may include apex Hₐ. Heart H may include heart wall H_{w}. Heart wall H_{w}, may include one or more of pericardium Hₚ, myocardium Hₘ and endocardium Hₑ. Heart wall H_{w} may include a septum between two cardiac atria. Heart wall H_{w} may include a septum between two cardiac ventricles.

Access hole Hₒ may extend through heart wall H_{w}. Access hole Hₒ may extend from exterior orifice Oₑ at a exterior side of heart wall Hw to interior orifice Oᵢ at an interior side of heart wall H_{w}. Exterior orifice Oₑ may be separated from interior orifice Oᵢ by access hole length hₒ.

Plug 100 may define central axis L_{c}. Central axis L_{c} may define radial direction R.

Plug 100 is shown inside delivery device 102. Delivery device 102 may include catheter 104. Delivery device 102 may include pusher member 106. Plug 100 may be loaded into delivery device 102 prior to insertion of delivery device 102 into heart H.

Plug 100 may include cap 108. Cap 108 may have a configuration, such as that shown in FIG. 1, in which cap 108 blocks or partially blocks a fluid such as blood B from contacting plug 100. Cap 108 may obstruct or partially obstruct the flow of blood B toward plug 100. Cap 108 may prevent or partially prevent plug 100 from expanding by absorption of blood B. Cap 108 may include material that may be viewed by non-visible-spectrum imaging instruments.

Cap 108 may be disposed at or near distal end 110 of plug 100. Cap 108 may be supported by longitudinal member 114. Longitudinal member 114 may be cannulated to accommodate a guidewire (not shown). Longitudinal member 114 may be present within cannula 116. Longitudinal member 114 may be drawn proximally to retract cap 108 through cannula 116 after placement of plug 100. When cap 108 is retracted, plug 100 may absorb blood B and expand in hole Ho.

Cap 108 may be biased to extend radially outward in radial direction R. Outer perimeter 118 of cap 108 may be deflected longitudinally against inner wall 112 of catheter 104 when plug 100 is loaded into delivery device 102 prior to insertion into heart H. Distal end 108 of delivery device 102 may be delivered into access hole Ho.

Cap 108 may have any suitable flexible structure. For example, cap 108 may include a mesh, a membrane, a membrane overlain mesh, a iris-like petals, a thin film or any other suitable structure. Cap 108 may be fixed to longitudinal member 114. Cap 108 may be rigidly fixed to longitudinal member 114. Cap 108 may be pivotably fixed to longitudinal member 114. Cap 108 may be fixed to longitudinal member 114 by an umbrella expansion/retraction mechanism. Cannula 116 may have a fluted distal end to receive cap 108 during retraction.

One or both of longitudinal member 114 and cannula 116 may be withdrawn through a cannula or cannulae (neither shown) of pusher member 106.

Plug 100 may have length hp. Length hp may be a length that is selected to be in proportion to access hole length ho. Plug 100 is illustrated as being about 100% of length ho.

Distal end 120 of catheter 104 is positioned a distance di distal from interior orifice Oi of access hole Ho. This places cap 108 distal interior orifice Oi.

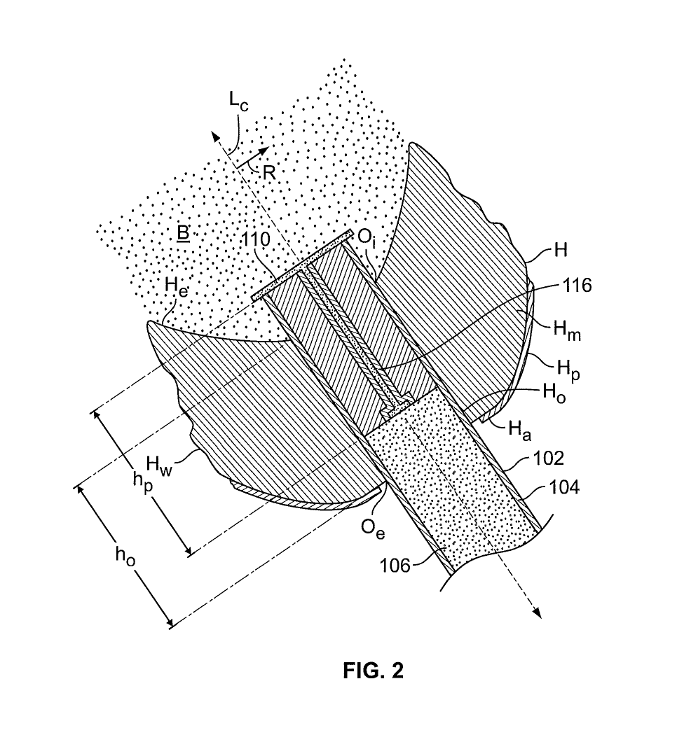

FIG. 2 shows that catheter 104 may been drawn proximally to deploy cap 108 in chamber Hc distal distal end 120 of catheter 104. Pusher member 106 may be held relative to heart H so that plug 100 remains in place during the drawing of catheter 104. Perimeter 118 of cap 108 may extend away from axis Lc in radial direction R. Distal end 110 of plug 100 may be maintained at a position distal of interior orifice Oi.

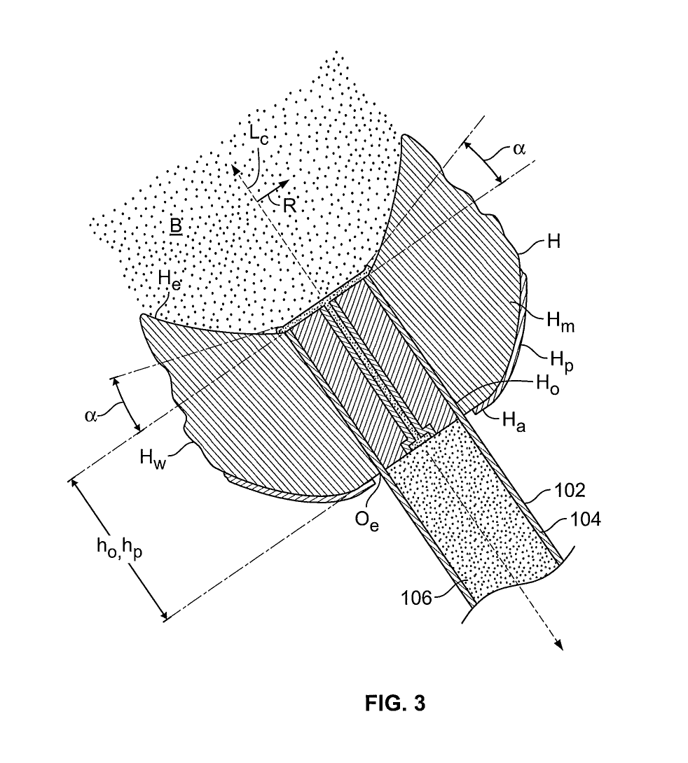

FIG. 3 shows that delivery device 102 may be drawn proximally to bring perimeter 118 of cap 108 in contact with heart wall Hw adjacent interior orifice Oi. Heart wall Hw adjacent interior orifice Oi may include tissue of endocardium He. As catheter 104 is drawn proximally, perimeter 118 of cap 108 may deflect to an angle α (from radial direction R) from contact with heart wall Hw. The deflection of perimeter 118 may signal that distal end 110 of plug 100 is positioned at or near interior orifice Oi. When perimeter 118 is at angle α, blood B may contact distal end 110 of plug 100.

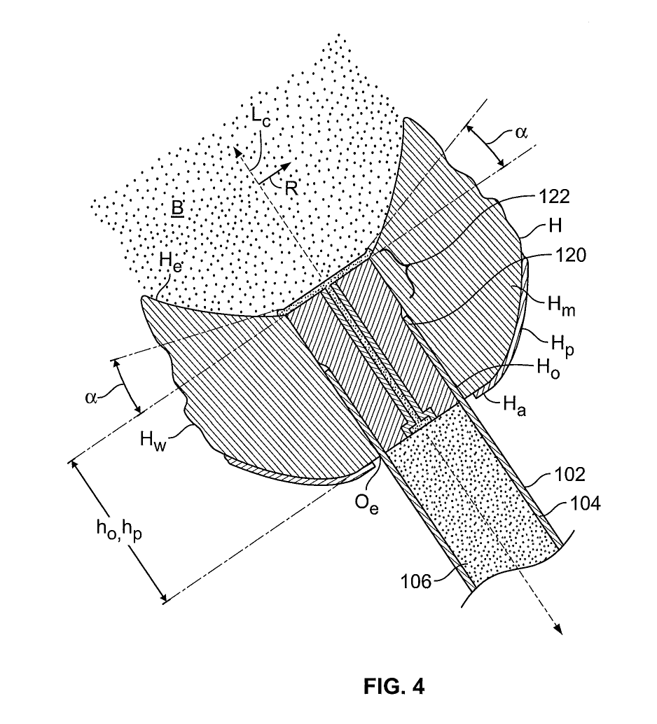

FIG. 4 shows that pusher member 106 may hold plug 100 in position in access hole Ho while catheter 104 is drawn proximally. Plug 100 may wick blood B into the interior and more proximal regions of plug 100. As distal end 120 of catheter 104 is drawn proximally, increasing region 122 of plug 100 expands to fill access hole Ho.

FIG. 5 shows plug 100 in access hole Ho. Cap 118 may be withdrawn through cannula 116. Longitudinal member 114 and cannula 116 may be withdrawn through pusher member 106 through cannula or cannulae (neither shown).

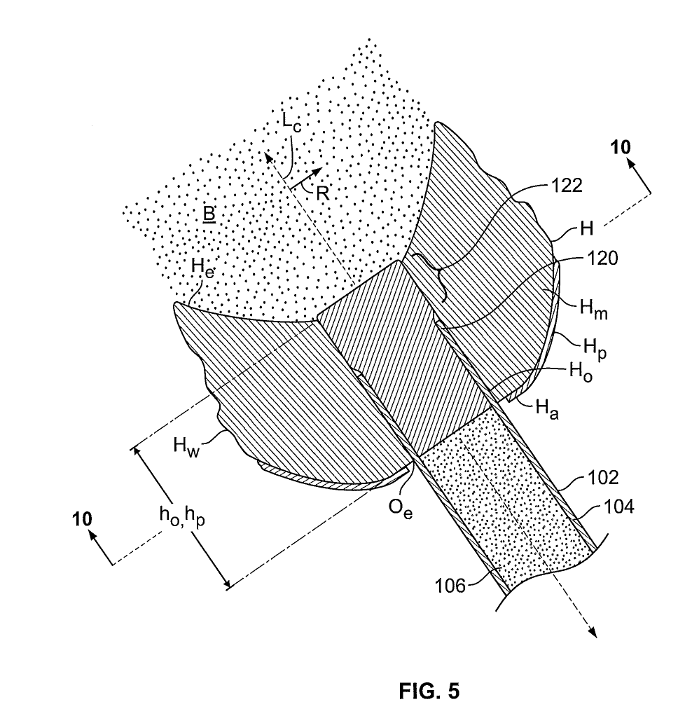

FIG. 6 shows that catheter 104 may be completely withdrawn from access hole Ho. Pusher member 106 may hold plug 110 in place while pusher member 106 is retracted from hole Ho. Plug 110 may hold itself in place by outward radial force on heart wall Hw while pusher member is retracted.

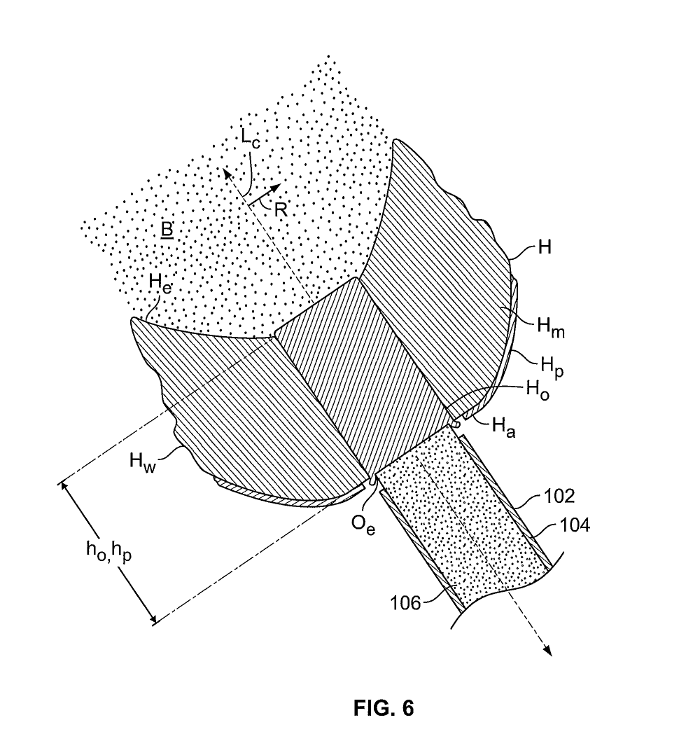

FIG. 7 delivery device separated from plug 100. Plug 100 is retained in access hole Ho. Plug 100 may be retained entirely by outward radial forces against heart wall Hw from absorption of blood B in plug 100. Plug 100 may be retained partially by outward radial forces against heart wall Hw from absorption of blood B in plug 100. Plug 100 may be retained partially by bonding to against heart wall Hw. The bonding may involve glue. The bonding may involve blood clotting. Plug 100 may be retained partially by engagement with heart H by one or more anchors such as barbs or tines.

FIG. 8 shows illustrative plug 800. Illustrative plug 800 may have one or more features in common with plug 100 (shown in FIG. 1). Plug 800 may include absorptive mass 802. Mass 802 may absorb fluid such as blood B (shown in FIG. 1). Absorption of the fluid may cause mass 802 to expand radially in direction R. Plug 800 may include veiling 804. Veiling 804 may surround mass 802. Veiling 804 may retain elements, such as fibers, of mass 802. Veiling 804 may provide traction on Hw (shown in FIG. 1) when mass 802 exerts pressure against heart wall Hw. The traction may retain or help retain plug 802 in heart H (shown in FIG. 1) when mass 802 exerts pressure against heart wall Hw.

Veiling 804 may be expandable. Veiling 804 may have a fixed diameter. When veiling 804 has a fixed diameter, veiling 804 may be folded into mass 802 during compression of mass 802 into the compressed state.

Veiling 804 or a separate veiling may cover distal face 810 of plug 800. Veiling 804 or a separate veiling may cover proximal face 812 of plug 800. A veiling on one or both of the terminal faces may retain elements of mass 802. Such a veiling may attenuate a rate of absorption of blood B by mass 802.

A veiling on proximal face 812 may be removable attached.

A veiling on distal face 810 may be removably attached. The veiling may attenuate the rate of absorption of blood B at distal face 810 of mass 80 when plug 800 is in heart chamber Hc.

Such a veiling may be retracted through a lumen (not shown) in plug 802. The lumen may be the lumen of a cannula. The lumen may be formed directly in mass 802. The lumen may be formed by use of a die in pressing mass 802 into the compressed state.

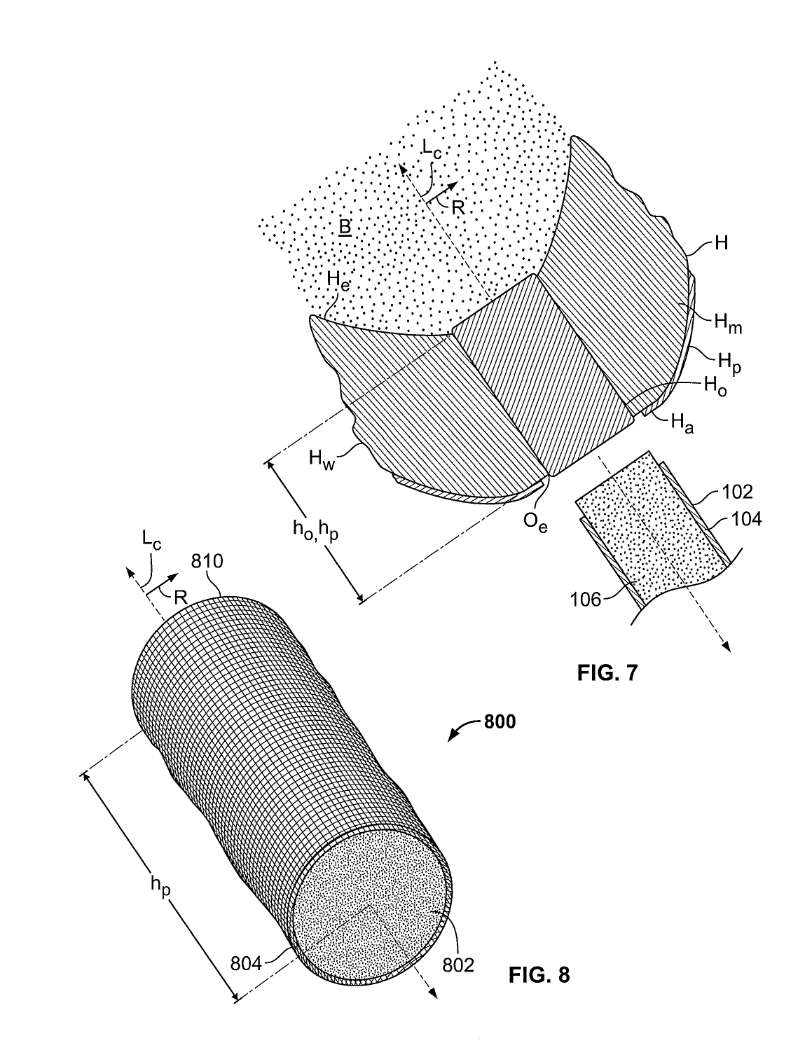

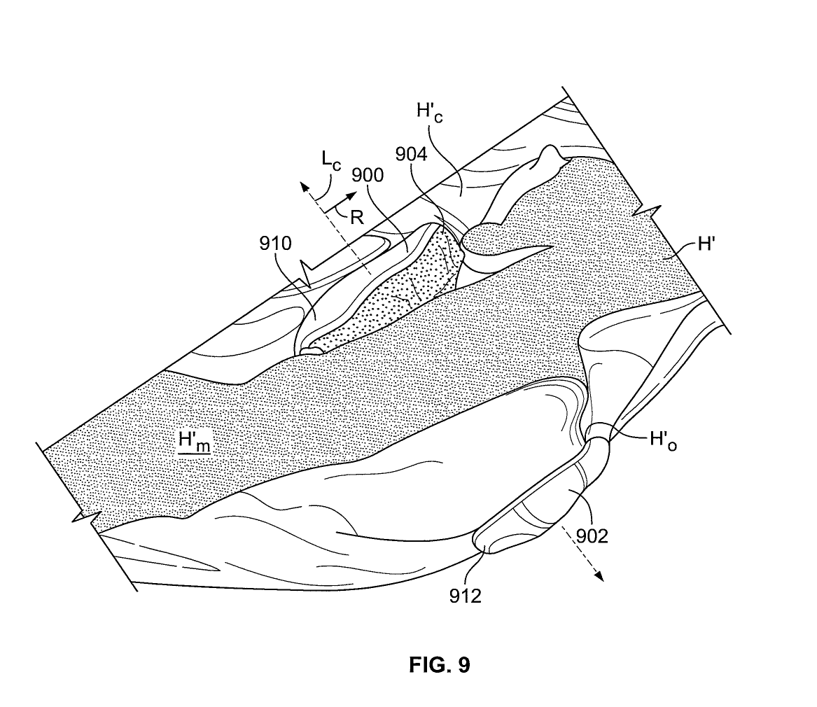

FIG. 9 shows plug 900 in heart H′. Plug 900 may have one or more features in common with one or both of plugs 100 and 800. Plug 900 is lodged in access hole H′o. Plug 900 may include mass 902. Plug 900 may include veiling 904. Mass 902 may exert radially outward pressure against heart wall H′w to hold plug 900 in access hole H′o against pressure from blood in chamber H′c while heart H′ is beating. Plug 900 extends longitudinally beyond heart wall H′w in both the distal and proximal directions.

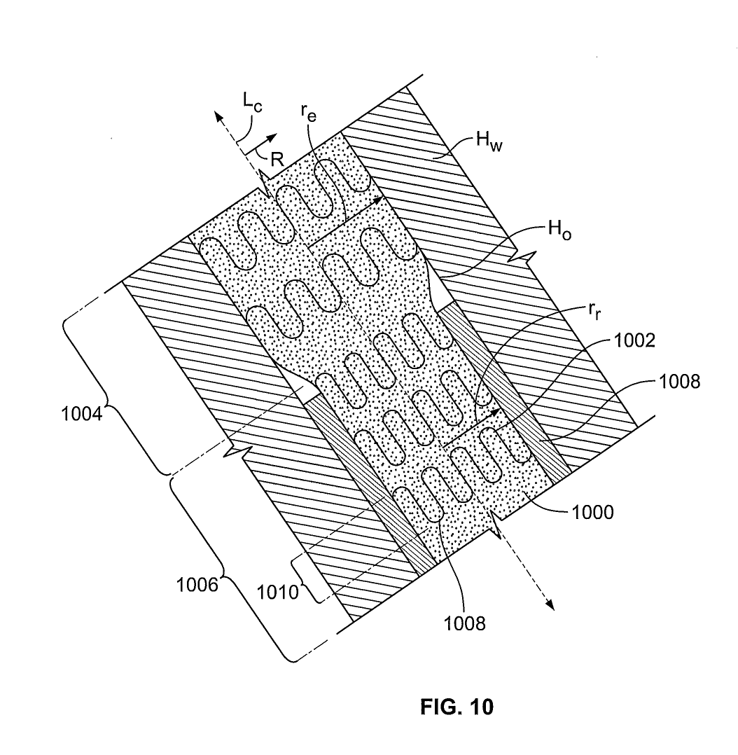

FIG. 10 shows illustrative plug 1000 in a view similar to that taken along lines 10-10 (shown in FIG. 5). Plug 1000 may have one or more features in common with one or more of plugs 100 (shown in FIG. 1), 800 (shown in FIG. 8) and 900 (shown in FIG. 9). Plug 1000 is shown in a state of deployment similar to the state of deployment shown in FIG. 5.

Plug 1000 may include a mass. Plug 1000 may include a veil. Plug 1000 may include marker 1002 that may be radiographically or acoustically viewable using medical imaging instrumentation. Marker 1002 may define a pattern. The pattern may be distributed about plug 1000 so that during deployment of plug 1000, expansion of section 1004 of plug 1000 to radius rₑ, relative to restriction of section 1006 by catheter 1008 to radius rᵣ, may be observed.

Marker 1002 may be disposed circumferentially about plug 1000. Marker 1002 may include one or more lobes such as lobe 1008 to allow marker 1002 to expand with plug 1000. Marker 1002 may include one or more “threads” such as thread 1010. Thread 1002 may be disposed around the circumference of plug 1000.

When section 1004 expands to radius rₑ, the lobes in that section may open. The lobes may open to render a thread partially or wholly circumferential or circular. This may provide a visual distinction relative to the lobes in section 1006.

One or more threads may be disposed helically around plug 1000. Marker 1002 may include discrete particles or objects in addition to or instead of threads.

Marker 1002 may be disposed about the mass of plug 1000. Marker 1002 may be disposed about a veiling of plug 1000. Marker 1002 may be printed on the mass. Marker 1002 may be woven into the mass. Marker 1002 may be printed on the veiling. Marker 1002 may be woven into the veiling.

FIG. 11 shows illustrative plug 1100. Plug 1100 may have one or more features in common with one or more of plugs 100 (shown in FIG. 1), 800 (shown in FIG. 8), 900 (shown in FIG. 9) and 1000 (shown in FIG. 10). Plug 1100 may include longitudinal member 1104. Longitudinal member 1104 may support distal cap 1106. Longitudinal member 1104 and distal cap 1106 may form a unitary member. Longitudinal member 1104 may support proximal cap 1108. Longitudinal member 1104 and proximal cap 1108 may form a unitary member. Longitudinal member 1104 and proximal cap 1108 may be joined by a releasable connection such as 1110. Connection 1110 may be a push-twist connection or any other suitable type of connection.

One or both of caps 1106 and 1108 may be biased in the longitudinal direction so that a perimeter of the caps presses against a terminal face of the corresponding cap.

FIG. 12 shows illustrative apparatus 1200 for closing an access hole in a heart. Apparatus 1200 may include delivery catheter 1202. Apparatus 1200 may include plug 1204. Apparatus 1200 may include pusher member 1206.

Delivery catheter 1202 may have one or more features in common with one or both of delivery catheter 104 (shown in FIG. 1) and delivery catheter 1008 (shown in FIG. 10).

Plug 1200 may have one or more features in common with one or more of plugs 100 (shown in FIG. 1), 800 (shown in FIG. 8), 900 (shown in FIG. 9), 1000 (shown in FIG. 10), and 1100 (shown in FIG. 11).

Pusher member 1206 may have one or more features in common with pusher member 106 (shown in FIG. 1).

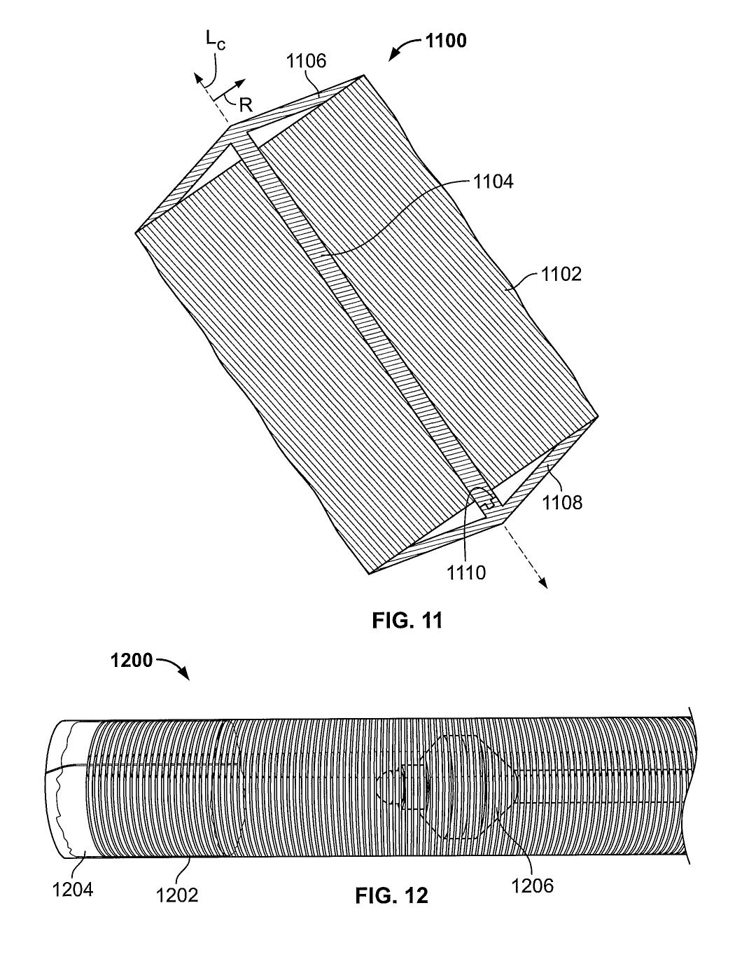

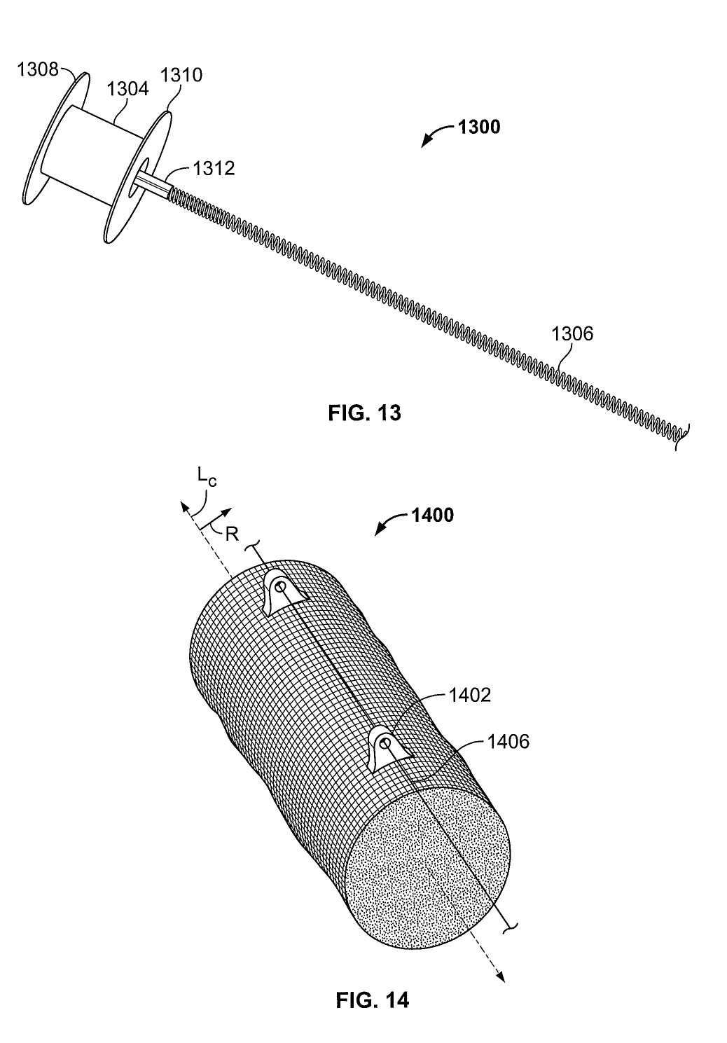

FIG. 13 shows illustrative apparatus 1300 for closing an access hole in a heart. Apparatus 1300 may include plug 1304. Apparatus 1300 may include pusher member 1306.

Plug 1304 may have one or more features in common with one or more of plugs 100 (shown in FIG. 1), 800 (shown in FIG. 8), 900 (shown in FIG. 9), 1000 (shown in FIG. 10), 1100 (shown in FIG. 11) and 1204 (shown in FIG. 12).

Pusher member 1306 may have one or more features in common with one or both of pusher member 106 (shown in FIG. 1) and pusher member 1206 (shown in FIG. 12).

Plug 1304 may include distal cap 1308. Plug 1304 may include proximal cap 1310. One or both of distal cap 1308 and proximal cap 1310 may have one or more features in common with one or more of cap 108 (shown in FIG. 1) and caps 1106 and 1108 (shown in FIG. 11).

Plug 1304 may be releasable engaged to pusher member at coupling 1312.

FIG. 14 shows illustrative plug 1400. Plug 1400 may have one or more features in common with one or more of plugs 100 (shown in FIG. 1), 800 (shown in FIG. 8), 900 (shown in FIG. 9), 1000 (shown in FIG. 10), 1100 (shown in FIG. 11), 1204 (shown in FIG. 12) and 1304 (shown in FIG. 13).

Plug 1400 may include one or more carriage loops such as carriage loop 1402. Carriage loop 1402 may receive guidewire 1406 for delivery into an access hole such as Ho in heart wall Hw. Plug 1400 may be delivered to the access hole using a delivery catheter such as 104. Plug 1400 may be delivered to the access hole without a delivery catheter.

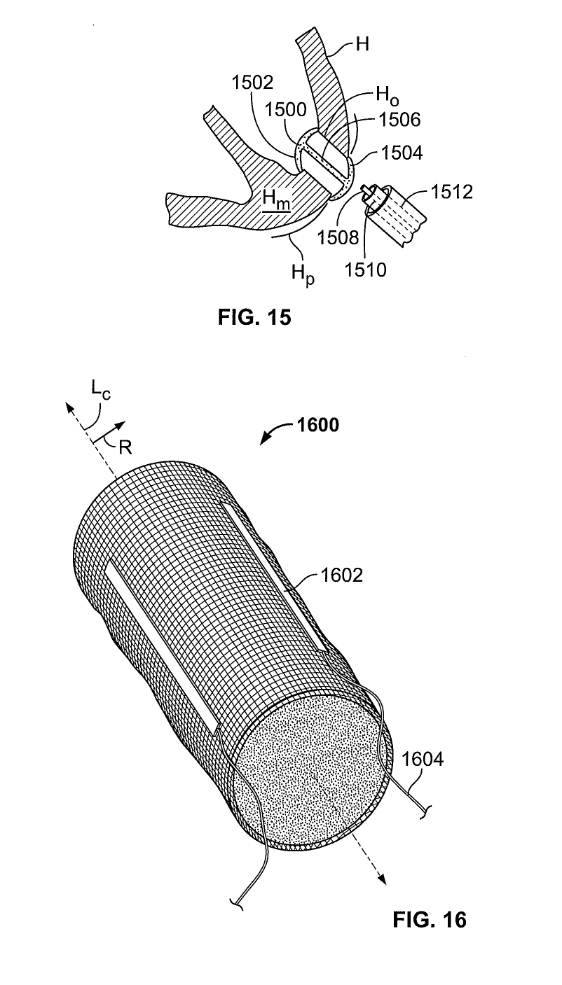

FIG. 15 shows illustrative plug 1500 in heart H (shown in FIG. 1). Plug 1500 may have one or more features in common with one or more of plugs 100 (shown in FIG. 1), 800 (shown in FIG. 8), 900 (shown in FIG. 9), 1000 (shown in FIG. 10), 1100 (shown in FIG. 11), 1204 (shown in FIG. 12), 1304 (shown in FIG. 13) and 1400 (shown in FIG. 14).

Plug 1500 may include distal cap 1502. Plug 1500 may include proximal cap 1504. One or both of caps 1502 and 1504 may have one or more features in common with one or more of caps 108 (shown in FIG. 1), 1106 and 1108 (shown in FIG. 11), and 1308 and 1310 (shown in FIG. 13).

One or both of the caps may include a septum. The septum may be self-sealing after penetration by a needle.

Plug 1500 may include shaft 1506. Shaft 1506 may include a mass. Plug 1506 may include a veiling. Shaft 1506 may be expandable by injection of a fluid through the needle. Shaft 1506 may expand to press against heart H by absorption of the fluid.

Plug 1500 may be delivered to access hole using one more of guidewire 1508, delivery catheter 1510 and access device 1512. Access device 1512 may have an internal lumen that has an internal valve for obstructing blood flow from heart H. The valve may permit passage of instrumentation, prostheses, closure devices and other suitable objects through access device 1512. Access device 1512 may be anchored to heart H. Access device 1512 may be configured to remain in access hole Ho without being anchored to heart H. Access device 1512 may be held in access hole Ho manually by a practitioner.

FIG. 16 shows illustrative plug 1600. Plug 1600 may have one or more features in common with one or more of plugs 100 (shown in FIG. 1), 800 (shown in FIG. 8), 900 (shown in FIG. 9), 1000 (shown in FIG. 10), 1100 (shown in FIG. 11), 1204 (shown in FIG. 12), 1304 (shown in FIG. 13), 1400 (shown in FIG. 14) and 1504 (shown in FIG. 15).

Plug 1600 may include one or more electrically conductive members such as 1602.

Electrically conductive member 1602 may be used to provide electrical pulses to heart wall H_{w} to change the contraction frequency. An electrically conductive member such as 1602 may be placed in direct contact with heart wall H_{w} to provide the electrical pulses. The energy may be supplied via a lead such as 1604 from a source (not shown). The energy may be supplied wirelessly from the source. The source may be programmable via a control panel (not shown). The source 110 may be incorporated into plug 1600. The source 110 may be implanted in a patient near plug 1600. Source 110 may be or include a pacing device.

An electrically conductive member may have a distal end that is placed in electrical communication with epidermal tissue on the body in which the heart is disposed.

An electrically conductive member may sense a native cardiac electric field. A signal corresponding to the field may be transmitted to the source. The signal may be transmitted via cable (not shown). The signal may be transmitted wirelessly. Plug 1600 may include one or more electrically conductive members that are wired to provide pulses to heart wall H_{w} and one or more electrically conductive members that are wired to transmit a native cardiac electric field signal to an electrocardiograph device.

Conductive member 1602 may be disposed about a mass of plug 1600. Conductive member 1602 may be disposed about a veiling of plug 1600. Conductive member 1602 may be printed on the mass. Conductive member 1602 may be woven into the mass. Conductive member 1602 may be printed on the veiling. Conductive member 1602 may be woven into the veiling.

Thus, apparatus and methods for closing an access hole have been provided. Persons skilled in the art will appreciate that the present invention can be practiced by other than the described embodiments, which are presented for purposes of illustration rather than of limitation.

The present invention is limited only by the claims that follow.

1. A heart plug comprising an absorbent mass that is configured to apply pressure to myocardial tissue in an access hole in a heart by absorption of fluid.

2. The heart plug of claim 1 having a length selected to match the length of the hole.

3. The heart plug of claim 1 having a diameter selected for occlusion of the hole and sliding clearance within a delivery catheter having an outside diameter sized for traversal of the hole.

4. The heart plug of claim 1 further comprising a growth factor surface treatment.

5. The heart plug of claim 1 wherein the mass comprises a matrix defining interstitial space for absorption of the fluid.

6. The heart plug of claim 5 further comprising an antibiotic pharmacological agent supported by the matrix.

7. The heart plug of claim 5 further comprising a polymerizing agent supported by the matrix.

8. The heart plug of claim 7 wherein the polymerizing agent is configured to provide a polymerized blood constituent in the interstitial space to give the plug elastic properties.

9. The heart plug of claim 5 wherein the matrix comprises fibrous matter.

10. The heart plug of claim 1 wherein the fibrous matter comprises cellulose.

11. The heart plug of claim 1 wherein the fibrous matter comprises cotton.

12. The heart plug of claim 1 wherein the mass comprises a porous polymer network.

13. The heart plug of claim 1 further comprising a non-removable cannula that extends through the mass and is configured to receive a guide wire.

14. The heart plug of claim 1 further comprising a non-removable cannula that extends through the mass and is configured to receive a guide wire.

15. The heart plug of claim 1 further comprising a carriage loop that extends away from an outer surface of the mass and is configured to receive a guide wire.

16. The heart plug of claim 1 further comprising a porous veiling about the mass, wherein the mass is configured to press the veiling against the myocardial tissue when the mass expands.

17. The plug of claim 16 wherein:
the mass has a dry diameter and a saturated diameter that is greater than the dry diameter;
the veiling has maximum diameter;
the hole has a diastolic diameter;
the saturated diameter is greater than the diastolic diameter; and
the maximum diameter is greater than the saturated diameter.

18. The plug of claim 16 wherein the porous veiling includes a bioabsorbable material.

19. The plug of claim 16 wherein the mass includes a bioabsorbable material.

20. The plug of claim 16 further comprising an anchor having a base that is affixed to the mass and an engagement end that is configured to engage the heart.

21. The plug of claim 20 wherein the engagement end is configured to atraumatically engage heart muscle.

22. The heart plug of claim 1 further comprising an imaging marker extending from a distal portion of the mass, the marker being configured to signal registration of the distal portion with an orifice at a distal end of the hole.

23. The heart plug of claim 22 wherein the imaging marker is radiopaque.

24. The heart plug of claim 22 wherein the imaging marker is selected for acoustic imaging.

25. The heart plug of claim 1 further comprising a non-thrombogenic cap extending, at a distal end of the mass, radially away from a central axis of the mass.

26. The plug of claim 25 wherein:
the mass has a first diameter when the mass is in a compressed state and a second diameter when the mass is in an expanded state; and
the cap has a cap diameter that is greater than the first diameter.

27. The plug of claim 26 wherein the cap is directly affixed to and extends away from a cannula extending, substantially along a central axis of the plug, from a proximal end of the plug to a distal end of the plug.

28. The plug of claim 26 wherein the cap diameter is about the same as the second diameter.

29. The plug of claim 25 further comprising a proximal cap extending, at a proximal end of the mass, radially away from the central axis.

30. The plug of claim 29 wherein the proximal cap is directly affixed to and extends away from a cannula extending, substantially along a central axis of the plug, from a proximal end of the plug to a distal end of the plug.

31. The plug of claim 25 wherein the cap includes a bioabsorbable material.

32. The plug of claim 25 further comprising a cannula extending from a proximal end of the plug to a distal end of the plug, the cannula being configured to receive a guidewire.

33. The plug of claim 24 further comprising:
an electrode; and
a lead;
wherein:
the veiling supports the electrode;
the electrode is configured to electrically engage the tissue; and
the lead has a first terminal connected to the electrode and a second terminal configured to provide communication to a device external to the heart.

34. A method for occluding a surgical access hole in a heart wall, the method comprising:
introducing into the access hole a plug that is configured to expand by fluid absorption to occlude the hole; and
positioning the plug adjacent myocardial tissue that is exposed in the hole; and
releasing the plug so that the plug, by exerting a traction on the tissue, resists being dislodged from the hole by systolic blood pressure.

35. The method of claim 34 wherein the positioning comprises placing the plug in direct contact with the tissue such that the traction is transmitted through the contact and not through an anchor.

36. The method of claim 35 wherein the placing comprises distributing contact between the plug and the tissue so that substantially all of the tissue is contacted by the plug.

37. The method of claim 35 wherein the placing comprises distributing contact between the plug and the tissue so that substantially all of the tissue in a length of the hole is contacted by the plug.

38. The method of claim 37 wherein the length defines a distal portion of the hole.

39. The method of claim 37 wherein the length defines a proximal portion of the hole.

40. The method of claim 34 wherein the introducing, the positioning and the releasing are completed in a period that has a duration that is less than a duration of time required for a clotting cascade to cause clotting material to sufficiently engage the plug with the tissue to resist the systolic pressure.

41. The method of claim 34 further comprising expanding the plug by injecting into the plug a fluid.

42. The method of claim 41 further comprising injecting a polymerizing agent into the plug, the agent being configured to polymerize a blood constituent in the plug to impart to the plug elastic properties.

43. The method of claim 42 wherein the injecting comprises transforming blood, absorbed in a matrix of the plug, into a solid.

44. The method of claim 34 wherein the systolic pressure is in a range from about 60 mm Hg to about 180 mm Hg.

45. The method of claim 34 wherein the introducing comprises delivering the plug percutaneously.

46. The method of claim 45 wherein the delivering comprises passing the plug through an epidermal incision having a length no greater than about one centimeter.

47. The method of claim 45 further comprising, after the releasing, permanently closing an epidermal incision.

48. The method of claim 34 wherein the releasing comprises deploying an absorbent core of the plug and a porous veiling of the plug, the porous veiling being disposed about the core, the core being configured to expand by absorption of the fluid and the veiling being configured to be displaced against the tissue by expansion of the core.

49. The method of claim 34 wherein the introducing comprises providing at a distal end of the plug a cap, extending radially away from a cylindrical axis of the plug, that is configured to retain fibers of the plug when the plug is in contact with the fluid.

50. The method of claim 34 wherein the introducing comprises advancing the plug along a guidewire that passes through the access hole.

51. The method of claim 34 wherein the plug is configured to seal, by absorption of the fluid, a lumen that is configured to translate along the wire.

52. The method of claim 34 wherein the releasing comprises disengaging a coupling between a proximal end of the plug and a distal end of a delivery wire that is not a guide wire.

53. The method of claim 34 wherein the positioning comprises advancing the plug distally in the hole, along a catheter lumen, using a pusher.

54. The method of claim 53 wherein the pusher is not coupled to the plug.

55. The method of claim 34 wherein the positioning comprises moving the plug proximally in the hole by moving a catheter proximally in the hole.

56. The method of claim 34 wherein the introducing comprises providing on a distal end of the plug an articulating radiopaque marker that mechanically signals detection of an internal orifice of the hole.

57. The method of claim 56 wherein the releasing comprises aligning a distal end of the plug with the internal orifice.

58. The method of claim 34 wherein the positioning comprises deploying a distal cap that extends, at a distal end of the plug, radially away from a central axis of the plug.

59. The method of claim 58 wherein the deploying comprises sliding the distal cap along a guide wire.

60. The method of claim 34 wherein the releasing comprises deploying a proximal cap that extends, at a proximal end of the plug, radially away from a central axis of the plug.

61. The method of claim 60 wherein the deploying comprises sliding the distal cap along a guide wire.

62. The method of claim 34 wherein:
the positioning comprises deploying a distal cap that extends, at a distal end of the plug, radially away from a central axis of the plug; and
the releasing comprises deploying a proximal cap that extends, at a proximal end of the plug, radially away from a central axis of the plug.

63. The method of claim 34 further comprising engaging a distal cap of the plug against endocardial tissue adjacent the hole.

64. The method of claim 34 further comprising delivering electrical current to a conductor held against the tissue by expansion of the plug.

65. The method of claim 64 wherein the delivering comprises controlling a heart rhythm.

66. The method of claim 34 further comprising receiving from a conductor held against the tissue by expansion of the plug electrical current corresponding to a heart rhythm.

67. The method of claim 34 further comprising receiving from the plug an electrical signal indicative of a position of the plug in the hole.

68. The method of claim 67 further comprising providing an electrical excitation to the plug before receiving the electrical signal.

69. The method of claim 34 wherein the releasing comprises deploying a distal end of the plug distal a distal orifice of the hole so that the distal end expands to a diameter greater than a diameter of the orifice.
